(19) United States
(12) Patent Application Publication
(10) Pub. No.: US 2010/0317740 A1
(43) Pub. Date: Dec. 16, 2010

(54) Method for Predicting Response to Tamoxifen

(75) Inventors: Emanuel F. Petricoin, Gainesville, VA (US); Lance A. Liotta, Bethesda, MD (US); Julia D. Wulfkuhle, Columbia, MD (US)

Correspondence Address: THE LAW OFFICE 0F GEOFFREY M. KARNY, 2152 BONAVENTURE DR., VIENNA, VA, 22181, US

(73) Assignee: GEORGE MASON INTELLECTUAL PROPERTIES, INC.

(21) Appl. No.: 12/669,833

(22) PCT Filed: Jul. 28, 2008
(86) PCT No.: PCT/US08/09105

§ 371 Date: Aug. 26, 2010

Related U.S. Application Data
(60) Provisional application No. 60/935,106, filed on Jul. 26, 2007.

(51) Int. Cl. A61K 31/135; G01N 33/53; A61P 35/04

(52) U.S. Cl. 514/648; 435/7.1

(57) ABSTRACT

This invention relates, e.g., to a method for predicting the response of a subject having, or at risk of developing, breast cancer to Tamoxifen therapy. The method comprises measuring the amount of phosphorylation at residues S70 of Bcl-2, Y992 of EGFR, and/or Y527 of Src in a suitable sample from the subject, wherein a statistically significantly elevated level of phosphorylation at one or more of the three residues compared to a baseline value indicates that the subject is likely to be responsive to Tamoxifen therapy.

This application claims the benefit of the filing date of U.S. Provisional application 60/935,106, filed Jul. 26, 2007 which is incorporated by reference herein in its entirety.

### BACKGROUND INFORMATION

Tamoxifen, a selective estrogen receptor modulator (SERM), is commonly used as a chemopreventive agent as well as an adjuvant therapy (therapy in addition to a primary treatment, e.g., to prevent a cancer from recurring or spreading to the other breast) for the treatment of advanced or recurrent breast cancer. Women with node negative breast cancer and with estrogen receptor on their tumors are eligible for Tamoxifen therapy. However, about 30-40% of eligible women do not respond to Tamoxifen therapy. Furthermore, Tamoxifen can elicit undesirable side effects, such as the risk of developing endometrial (uterine) cancer or uterine sarcomas, liver toxicities, etc. It would be desirable to have a method for stratifying patients, to distinguish responders from non-responders. In this manner, one could identify a class of subjects who would benefit from Tamoxifen therapy, and could avoid subjecting non-responders to the deleterious side-effects associated with Tamoxifen therapy without its benefit. Furthermore, many subjects develop resistance to Tamoxifen after time, and exhibit recurrent breast cancer following relapses. Often, after Tamoxifen resistance sets in, agonistic (rather than inhibitory) effects of Tamoxifen take over, leading to the progression of breast cancer tumors, secondary malignancies (e.g., uterine cancers), etc. It would be desirable to identify a class of subjects (responders) in whom the agonistic effects of Tamoxifen are reduced or eliminated, and who thus exhibit longer progression-free survival and/or post-relapse survival than non-responders. In this manner, one could identify the subclass of subjects who would benefit from Tamoxifen administration in such a recurrent setting, rather than being subjected to such agonistic effects.

In addition, by identifying a class of subjects that are resistant to Tamoxifen treatment, one can identify candidates for more aggressive therapies for breast cancer, such as aromatase inhibitors (AI). Aromatase inhibitors are associated with moderate to severe bone loss, so giving all women AI therapy would be unacceptable. A biomarker that could discriminate outcome and response to a less detrimental therapy, such as Tamoxifen therapy, would be of great benefit for this example cohort.

Gene expression analysis has provided the means to derive prognostic signatures for some outcomes. However, the analysis of the many genes in gene expression analysis is complex, and generally involves the use of algorithms and extensive computer analysis and does not reflect the activated or functional state of the protein drug targets. It would be desirable to identify biomarkers that can predict response or resistance to Tamoxifen treatment, particularly in the recurrent setting. Protein biomarkers (e.g., phosphoprotein markers) would be highly attractive due to their ease of clinical implementation and economics of cost.

### DESCRIPTION OF THE DRAWINGS

FIG. 1 shows a Kaplan-Meier curve for Progression-Free-Survival (PFS), in months, as a function of phosphorylation of PKCct S657.

FIG. 2 shows a Kaplan-Meier curve for Progression-Free-Survival (PFS), in months, as a function of phosphorylation of EGFR (EGF receptor) Y992.

FIG. 3 shows a Kaplan-Meier curve for Progression-Free-Survival (PFS), in months, as a function of phosphorylation of Bcl-2 S70.

FIG. 4 shows a Kaplan-Meier curve for Progression-Free-Survival (PFS), in months, as a function of phosphorylation of Src Y527.

FIG. 5 shows a Kaplan-Meier curve for Post-Relapse-Survival (PRS), in months, as a function of phosphorylation of EGFR Y992.

FIG. 6 shows a Kaplan-Meier curve for Post-Relapse-Survival (PRS), in months, as a function of phosphorylation of Src Y527.

FIG. 7 shows a Kaplan-Meier curve for Post-Relapse-Survival (PRS), in months, as a function of phosphorylation of Bcl-2 Y992.

### DESCRIPTION OF THE INVENTION

The present invention relates, e.g., to a method for determining (predicting) whether a subject (patient) having breast cancer (e.g., recurrent breast cancer), or at risk for developing breast cancer, is likely to be responsive (susceptible, amenable) to Tamoxifen therapy. The Examples herein demonstrate that responders to Tamoxifen therapy during recurrent breast cancer exhibited a statistically significantly increased amount of phosphorylation at particular residues of three phosphoprotein markers—Bcl-2 S70, EGFR Y992, and Src Y527—than did non-responders (patients in whom the cancer progressed). That is, the total amount of these phosphorylated isoforms in samples of tumor epithelial cells from the subjects was statistically significantly increased in responders compared to the amounts in non-responders, as measured by progression-free survival (PFS) and/or post-relapse survival (PRS). “PFS” refers to the length of time during and after treatment in which a patient living with a disease does not get worse. “PRS” refers to the length of time between the time a patient has recurred or relapsed with a disease and the time of death. These findings are in contrast to the amounts of phosphorylation at specific residues of about 19 other key proteins regulating apoptosis, proliferation, and survival signaling, for which mean comparison revealed no significant differences between the responder and non-responder categories as measured by PFS and/or PRS. Thus, the three noted phosphoprotein biomarkers provide useful information for rational therapeutic selection of Tamoxifen and improved response rates, e.g. in a recurrent setting.

Advantages of a method of the invention include that the method is rapid and inexpensive, and can be performed with non-invasive sampling techniques.

One aspect of the invention is a method for predicting the response (e.g., as measured by PFS and/or PRS) of a subject having breast cancer (e.g. having recurrent, non-metastasizing estrogen receptor positive (ER⁺) breast cancer), or being at risk for developing breast cancer, to treatment with Tamoxifen (sometimes referred to herein as Tamoxifen therapy). The method comprises measuring the amount (level) of phosphorylation at residue S70 of Bcl-2, residue Y992 of EGFR, and/or residue Y527 of Src in a suitable sample from the subject, compared to a baseline value. The method may comprise measuring the amount of Bcl-2 that is phosphorylated at residue S70, the amount of EGFR that is phosphorylated at residue Y992, and/or the amount of Src that is phosphorylated at residue Y527 in a suitable sample from the subject per cell in the sample. The sample generally contains a tissue or cell from the subject. A typical sample is from a tumor biopsy. An elevated level (e.g. a significantly elevated level) of phosphorylation at one or more (e.g., two or more, or all three) of these residues, compared to a baseline value, indicates that the subject is likely to be responsive to therapy with Tamoxifen (e.g. as measured by PFS or PRS).

In one embodiment of the invention, the baseline value to which the levels of phosphorylation are compared is expressed with regard to negative and positive reference standards. In this embodiment, the amount of phosphorylation at each of the residues compared to the amount in negative and in positive reference standards, wherein (1) a statistically significantly elevated level of phosphorylation of one or more of Bcl-2(S70), EGFR(Y992) or Src(Y527) compared to the negative reference standard, or a level that is statistically the same as the positive reference standard, indicates that the subject is likely to be responsive to Tamoxifen therapy (e.g. as measured by PFS or PRS); and (2) a statistically significantly reduced level of phosphorylation of one or more of Bcl-2(S70), EGFR(Y992) or Src(Y527) compared to the positive reference standard, or a level that is statistically the same as the negative reference standard, indicates that the subject is likely to be non-responsive to Tamoxifen therapy (e.g. as measured by PFS or PRS).

Another aspect of the invention is a method for determining (predicting) the prognosis of a subject as above, which is receiving Tamoxifen treatment. The method comprises measuring the amount of phosphorylation at Bcl-2 residue S-70, EGFR residue Y992, and/or Src residue Y527 in a suitable sample from the subject (e.g., the amount of each phosphoprotein per cell in the sample), compared to a baseline value. If the amount(s) of phosphorylation are significantly elevated compared to the baseline value, the subject is likely to have a good prognosis. By “good prognosis,” as used herein, is meant a greater than about 10% decrease in the time to recurrence following treatment compared to the expected mean recurrence rate for a treated patient. A “poor” prognosis refers to a greater than about 10% increase in the time to recurrence compared to the expected mean recurrence rate.

Another aspect of the invention is a method for determining a therapeutic treatment regimen for a subject as above, which is based upon the subject's expected response or lack of response to treatment with Tamoxifen (e.g., as measured by PFS or PRS), comprising determining an expected response or non-response to Tamoxifen by a method of the invention, and selecting an appropriate treatment for a subject having the expected response. Another aspect of the invention is a method for treating a subject as above, comprising determining if the subject is likely to respond or not respond to Tamoxifen by a method of the invention, and treating the subject accordingly.

A subject that is determined by a method of the invention to be likely to be responsive to Tamoxifen treatment (e.g. as measured by PFS or PRS) can be treated with Tamoxifen. A subject that is determined by a method of the invention not to be likely to be responsive to Tamoxifen treatment (e.g. as measured by PFS or PRS) can be treated with a treatment other than Tamoxifen treatment. For example, the subject can be treated with any method falling under the art-recognized “standard of care” other than Tamoxifen treatment. Suitable treatments include one or more of treatment with an aromatase inhibitor, many of which will be evident to a skilled worker, with radiation, or with any of a variety of chemotherapeutic agents, such as those listed in Table 1.

TABLE 1

| | |
|---|---|
| Mechanism of action | Class (chemotherapeutic agent, drug names) |
| Alkylating agents | Nitrogen mustards: (Chlorambucil, Chlormethine, |
| | Cyclophosphamide, Ifosfamide, Melphalan). Nitrosoureas: |
| | (Carmustine, Fotemustine, Lomustine, Streptozocin). Platinum: |
| | (Carboplatin, Cisplatin, Oxaliplatin, BBR3464). Busulfan, |
| | Dacarbazine, Mechlorethamine, Procarbazine, Temozolomide, |
| | ThioTEPA, Uramustine |
| Antimetabolites: | Folic acid: (Methotrexate, Pemetrexed, Raltitrexed). Purine: |
| | (Cladribine, Clofarabine, Fludarabine, Mercaptopurine, |
| | Tioguanine). Pyrimidine: (Capecitabine). Cytarabine, Fluorouracil, |
| | Gemcitabine |
| Plant alkaloids: | Taxane: (Docetaxel, Paclitaxel). Vinca: (Vinblastine, Vincristine, |
| | Vindesine, Vinorelbine). |
| Cytotoxic/antitumor | Anthracycline family: (Daunorubicin, Doxorubicin, Epirubicin, |
| antibiotics: | Idarubicin, Mitoxantrone, Valrubicin). Bleomycin, Hydroxyurea, |
| | Mitomycin |
| Topoisomerase inhibitors: | Topotecan, Irinotecan, Podophyllum: (Etoposide, Teniposide). |
| Monoclonal antibodies: | Alemtuzumab, Bevacizumab, Cetuximab, Gemtuzumab, |
| | Panitumumab, Rituximab, Trastuzumab |
| Photosensitizers: | Aminolevulinic acid, Methyl aminolevulinate, Porfimer sodium, |
| | Verteporfin |
| Other: | Alitretinoin, Altretamine, Amsacrine, Anagrelide, Arsenic trioxide, |
| | Asparaginase, Bexarotene, Bortezomib, Celecoxib, Denileukin |
| | diftitox, Erlotinib, Estramustine, Gefitinib, Hydroxycarbamide, |
| | Imatinib, Pentostatin, Masoprocol, Mitotane, Pegaspargase, |
| | Tretinoin |
| Hormones | Progesterones |

As used herein, the singular forms “a,” “an” and “the” include plural referents unless the context clearly dictates otherwise. For example, “an” aromatase inhibitor, as used above, includes two or more aromatase inhibitors.

Another aspect of the invention is a method for treating a subject as above, comprising administering to the subject an effective amount of Tamoxifen if a suitable sample from the subject is shown to exhibit a statistically significantly elevated level of phosphorylation at residue S70 of Bcl-2, residue Y992 of EGFR, and/or residue Y527 of Src, compared to a baseline value.

Another aspect of the invention is in a method for treating a subject as above, the improvement comprising determining that a suitable sample from the subject exhibits a statistically significantly elevated level of phosphorylation at residue S70 of Bcl-2, residue Y992 of EGFR, and/or residue Y527 of Src, compared to a baseline value, and then administering an effective amount of Tamoxifen.

Another aspect of the invention is a method for monitoring the effectiveness of treating a subject as above with Tamoxifen, comprising measuring the amount of phosphorylation at residue S70 of Bcl-2, residue Y992 of EGFR, and/or residue Y527 of Src in a suitable sample from the subject, compared to the amount of phosphorylation of the subject before Tamoxifen treatment was initiated, or at the time at which Tamoxifen treatment was initiated, wherein a statistically significantly reduced level of phosphorylation of one or more of Bcl-2(S70), EGFR(Y992) or Src(Y527) compared to the level of phosphorylation before the treatment began, or at the time the treatment was initiated, indicates that the subject is becoming (or has become) non-responsive to the Tamoxifen therapy.

In methods of the invention, the amount of phosphorylation at one or more of the residues can be detected by measuring the amount of reactivity of an antibody specific for the phosphorylated isoform of residue S70 of Bcl-2, residue Y992 of EGFR, and/or residue Y527 in the sample; and/or the subject can be human.

Another aspect of the invention is a kit for predicting the response to Tamoxifen of a subject as above, comprising reagents for measuring the amount of phosphorylation at residue S70 of Bcl-2, residue Y992 of EGFR, and/or residue Y527 of Src. The components of the kit may, optionally, be packaged in one or more containers.

Another aspect of the invention is a method comprising (a) obtaining a tissue sample (e.g. from a subject as above); (b) obtaining data regarding the levels of phosphorylation of one or more of residue S70 of Bcl-2, residue Y992 of EGFR, and/or residue Y527 of Src in the sample; (c) comparing the levels of phosphorylation of the residue(s) in the sample to those in a control population (e.g., a population of subjects having breast cancer than is known to be responsive to Tamoxifen treatment); and (d) providing a report that ranks the phosphorylation level(s) in the subject to the levels on the control population.

The present invention provides methods for selecting subjects for treatment with Tamoxifen, comprising determining the presence or amount of phosphorylated Bcl-2 S70, EGFR Y992, and/or Src Y527 in suitable samples obtained from subjects. The amino acid residue numbering of these three proteins is well-known and can be determined routinely, or can be downloaded from various known databases. See, e.g., the world wide web site ncbi.nlm.nih.gov. The numbering of Bcl-2 amino acid residues is in accordance with the known Bcl-2 sequences (e.g., Tsujimoto et al. (1986) Proc. Natl. Acad. Sci. U.S.A. 83, 5214-5218); the numbering of EGFR amino acid residues is in accordance with the known EGFR sequences (e.g., Ullrich et al. (1984) Nature 309, 418-425); and the numbering of Src amino acid residues is in accordance with the known Src sequences (e.g., P. Deloukas (2001) Nature 414, 865-87).

A “subject,” as used herein, includes any animal that has, or is at risk for developing, a breast cancer. A method of the invention may be used to determine the responsiveness of a subject to Tamoxifen treatment at any stage of breast cancer. For example, the subject may be at risk for developing breast cancer, but asymptomatic, in which case Tamoxifen treatment would be prophylactic. Subjects at risk for developing breast cancer include, e.g., subjects that harbor mutations in the BRCA1 or BRCA 2 genes, subjects with a family history of breast cancer or ovarian cancer, or that are morbidly obese, have a history of smoking, etc. A number of studies have indicated that prophylactic use of Tamoxifen can be useful for preventing or inhibiting the development of breast cancer in subjects that are at risk for the disease. Other suitable subjects include subjects having early stage breast cancer, in which case the Tamoxifen treatment would, e.g., prevent growth and/or metastasis of the cancer; subjects in remittance from (recurring) breast cancer, in which case the Tamoxifen treatment would, e.g., prevent recurrence of the cancer or spread to the other breast; and subjects having active recurrent breast cancer, in which case the Tamoxifen treatment would, e.g., inhibit growth and/or spread of the cancer. In one embodiment of the invention, the subject (e.g., patient) has been diagnosed as having node-negative and estrogen receptor (ER) positive breast cancer, which is recurrent. In one embodiment, the cancer is non-metastasizing. In one embodiment, the subject has recurrent, non-metastasizing ER⁺ breast cancer). The subject may be progesterone receptor (PrR) positive.

Other diseases or conditions that can be treated with Tamoxifen are well-known in the art and include, e.g., ovarian cancer, colorectal cancer and pancreatic cancer. Suitable subjects that can be tested for responsiveness to Tamoxifen by a method of the invention thus also include subjects having one of those diseases or conditions.

Suitable subjects include laboratory animals (such as mouse, rat, rabbit, or guinea pig), farm animals, and domestic animals or pets (such as a cat or dog). Non-human primates and, preferably, human patients, are included. Although the subjects are generally female, males (e.g. men) can also have breast cancer and can be subjected to a method of the invention.

Sources of “suitable samples” will be evident to a skilled worker. Suitable samples comprise tissues or cells from a subject which contain one or more of the three biomarkers, e.g., biopsy or other tissue or cell samples (such as a tumor biopsies), primary tissue, blood cells, serum, body fluids, low-molecular weight fraction, etc. When assessing the value of prophylactic Tamoxifen treatment for a subject that does not yet exhibit tumors, suitable samples include, e.g., blood cells, or tissue biopsy of normal tissue, such as normal breast epithelium.

As used herein, a subject that is “non-responsive” to Tamoxifen treatment is one exhibiting a 10% or greater reduction in survival following treatment with Tamoxifen compared to the median value of a similar population of subjects that is treated with Tamoxifen, as measured by progression-free survival (PFS) or post-relapse survival (PRS). Similarly, a subject that is “responsive” to Tamoxifen treatment is one exhibiting 10% or greater increase in survival following treatment with Tamoxifen, compared to the median value of a similar population of subjects that is treated with Tamoxifen, measured as above.

A “baseline value,” as used herein, refers to the level of phosphorylation at a given amino acid residue of a protein (e.g., on the amino acid side chain) in a subject that is not responsive to Tamoxifen, or the median value of the levels of phosphorylation a control population of subjects that are not responsive to Tamoxifen. An increase in the amount of phosphorylation of a protein (e.g., an increase in the total amount per cell of a phosphoprotein isoform of interest) can reflect an increased frequency of phosphorylations at the amino acid residue. In general, the total amount of protein that is phosphorylated at the noted amino acid residue is measured, per sample or per cell in the sample.

In one embodiment, the baseline value is determined by preparing positive and negative reference standards derived from tissue culture cells.

To generate a “negative” reference standard, one can first process cells obtained from a biopsy specimen (such as a human biopsy specimen) from a subject (or a pool of subjects) that is known to be non-responsive to Tamoxifen. Protein extracts can be prepared from the tissue and the level of phosphorylation (or range of values) at the phospho-endpoints of interest determined as described herein. The median value of such samples can serve as a negative reference standard.

To generate a “positive” reference standard, one can process cells from a comparable tissue from a subject (or a pool of subjects) that is known to be responsive to Tamoxifen. Protein extracts can be prepared from the tissue and the level of phosphorylation (or range of values) at the phospho-endpoints of interest determined as described herein. The median value of such samples can serve as a positive reference standard.

However, using such tissue from subjects as a clinical diagnostic reference standard is generally not practical on a routine basis. Instead, it is preferable to generate negative and positive reference standards by using lysates from cells in culture, and establishing a cut-point value by a direct comparison of the cell culture lysates to a true positive (e.g. endpoint values derived from responsive subjects as described above) and true negative (e.g. endpoint values derived from non-responsive control subjects as described above). To accomplish this, one can first screen a variety of cells in culture, either primary cells or, preferably, cell lines (e.g., any of a variety of well-known Tamoxifen resistant or Tamoxifen sensitive breast cancer cell lines).

The cells in culture can be propagated directly, under conventional conditions, so that, e.g., Bcl-2, EGFR and/or Src are not phosphorylated or are phosphorylated to a minimal degree; or they can be incubated under conventional conditions with a suitable mitogen that will globally activate signaling networks, such as pervanadate, or a growth factor, such as epidermal growth factor (EGF).

Protein extracts are then prepared from the various cell lines, which have been incubated under the various conditions, using conventional procedures; and the level of phosphorylation at the phospho-endpoints of interest determined as described herein, and compared directly to the true positive and true negative clinical samples as a bridging experiment. In this way, one can establish conditions such that particular cells, cultured under particular defined conditions (stimulated or not), express an amount of phosphorylation of the phosphoprotein isoforms of the invention that is directly comparable to those of a subject that is responsive to Tamoxifen or that is not responsive to Tamoxifen. Utilizing the cut-point values derived from median values of known true clinical positives and negatives, and bridging these values to a cell line reference standard can then provide a “positive reference standard” or a “negative reference standard,” respectively.

Alternatively, a baseline value can be the level of phosphorylation in a purified sample of the analyte (e.g., one or more of the phosphorylated protein isoforms of the invention) of known concentration.

In one embodiment of the invention, the level of phosphorylation of a marker of the invention can be characterized within a range of phosphorylation levels within a given affected population cohort (e.g., breast cancer patients as above). Relative amounts of a given patient within the cohort can be classified and characterized in relation to the entire population distribution and categorized as high or low (or average) as compared to the rest of the patient population values. A patient that falls within the top quartile of the population can be considered to be likely to be responsive to Tamoxifen treatment.

The baseline values or ranges may be determined by a variety of conventional procedures that will be apparent to a person of ordinary skill. Baseline values may be determined, e.g., based on published data; retrospective studies of patients that have responded, or failed to respond to, Tamoxifen therapy; and other information as would be apparent to a person of ordinary skill implementing the methods of the invention. The baseline values may be selected using statistical tools that provide an appropriate confidence interval so that measured levels that are higher than the baseline value can be accepted as being predictive of a positive response to Tamoxifen therapy.

For each protein whose level of phosphorylation is determined, the value can be normalized, e.g., to the total protein in the cell; or to the amount of a constitutively expressed protein (from a housekeeping gene), such as actin; or the amount of a phosphoprotein may be compared to the amount of its non-phosphorylated counterpart.

A “statistically significantly elevated” level of phosphorylation (compared to a baseline value or negative reference standard) is a level whose difference from the baseline value or negative reference standard is statistically significant, using statistical methods that are appropriate and well-known in the art, generally with a probability value of less than five percent chance of the change being due to random variation. For example, the phosphorylation of the Bcl-2 S70, EGFR Y992, or Src Y527 residues in a subject that is responsive to Tamoxifen may range from about a 50% increase to 10-fold higher (e.g. 5-fold higher), or more, than the level observed in a subject that is non-responsive. A “significantly reduced” level of phosphorylation, as used herein, is a comparable difference from a positive reference standard, or from a subject that is non-responsive to Tamoxifen.

The level of phosphorylation of a given amino acid residue can be measured qualitatively or quantitatively. The amount (quantity) of phosphorylation at a given residue may be higher than is observed at the same residue in a control sample (a baseline value). That is, it may be hyperphosphorylated. In addition to hyperphosphorylation as a detection threshold, the presence or absence of phosphorylation at the noted residues can also be utilized. Alternatively, a qualitative scale (such as a scale of 1 to 5) can be used.

Methods for measuring the level of phosphorylation at an amino acid residue are conventional and routine. In one embodiment, the measurement relies on the existence of sets of antibodies that are specific for either the non-phosphorylated or the phosphorylated forms of a particular amino acid residue of interest in the context of a protein of interest (such as phosphorylation of the noted residues of Bcl-2, EGFR and/or Src). Such antibodies are commercially available or can be generated routinely, using conventional procedures. In one embodiment, a synthetic peptide comprising an amino acid of interest from a protein of interest (either in the non-phosphorylated or phosphorylated form) is used as an antigen to prepare a suitable antibody. The antibody can be polyclonal or monoclonal. Antibodies are selected and verified to detect only the phosphorylated version of the protein but not the non-phosphorylated version of the native or denatured protein, and vice-versa.

Such antibodies can be used in a variety of ways. For example, in a method referred to as a reverse phase protein microarray assay (RPMA), one prepares whole cell lysates from patient samples and attaches (e.g., spots) them in an array format onto a suitable substrate, such as nitrocellulose strips or glass slides. Preferably, the proteins in the samples are denatured before spotting. In one embodiment, the lysates are spotted at serial dilutions, such as two-fold serial dilutions, to provide a wide dynamic range. Suitable controls, such as negative or positive controls (e.g., controls for base line values), can be included. In another embodiment, no more than one dilution of a lysate is spotted, but a set of calibrants comprising a range of defined amounts of the analytes in a suitable diluent (e.g., a cell or tissue lysate or a bodily fluid) is also spotted on each substrate. The analytes in the lysates and the calibrant are detected with a detectable moiety that has a large dynamic range (e.g. at least two orders of magnitude). Such a procedure obviates the need to spot derail dilutions in order to provide a wide dynamic range. Each array is then probed with a suitable detectable antibody, as described above, to determine and/or to quantitate amino acid residue(s) in the various proteins of interest that are phosphorylated. Methods for immuno-quantitation are conventional. For a further discussion of the method of RPMA, see, e.g., Nishizuka et al. (2003) Proc. Natl. Acad. Sci. 100, 14229-14239.

Other suitable assays employing such antibodies to assess the level and/or degree of phosphorylation at a residue of interest include, e.g., Western blots, ELISA assays, immunoprecipitation, mass spectroscopy, and other conventional assays. Suitable methods include those that can detect the phosphoprotein in a very small sample (e.g. about 200 cells). Alternatively, methods can be used that are suitable for a large sample size (e.g. about 20,000-25,000 cells).

Assays to measure the presence and/or amount of phosphorylated residues can be readily adapted to high throughput formats, e.g. using robotics.

The measured amounts of phosphorylation from a sample can be compared to baseline values (e.g., positive or negative reference standards) by any of a variety of art-recognized methods. For example, reference standards can be assayed simultaneously with samples from a subject, or the amount of phosphorylation in a sample from a subject can be compared to independently measured or derived reference standards.

One aspect of the invention is a method for determining if a subject is likely to be responsive to Tamoxifen treatment (e.g. as measured by PFS or PRS). A subject that is “likely to be responsive,” as used herein, is a subject that is more likely (e.g., greater than about 50% more likely) than a control group from the general population to be responsive to Tamoxifen.

One aspect of the invention is a method for treating a subject having recurrent but not metastasizing breast cancer, comprising (1) measuring the amount of phosphorylation at Bcl-2 S70, EGFR Y992, and/or Src Y527 in a suitable sample from the subject and, if the levels of phosphorylation compared to a baseline value suggest that the subject is likely to be responsive to Tamoxifen therapy (e.g. as measured by PFS or PRS), (2) administering an effective amount of Tamoxifen to the subject. Methods for administering Tamoxifen to a subject are conventional and well-known in the art. An “effective” amount of Tamoxifen is an amount that is sufficient to elicit a measurable amount of a therapeutic activity. If the levels of phosphorylation compared to a baseline value suggest that the subject is not likely to be responsive to Tamoxifen therapy, forms of therapy other than Tamoxifen therapy, including more aggressive forms of therapy, can be administered. These include, e.g., the administration of an effective amount of an aromatase inhibitor, radiation treatment, or treatment with any of a variety of chemotherapeutic agents, such as those listed in Table 1.

Another aspect of the invention is a method for monitoring the effectiveness of a treatment of a breast cancer with Tamoxifen. It is expected that level of phosphorylation at the residues discussed herein will remain at approximately the same level if the subject remains responsive to Tamoxifen. However, if the subject begins to develop resistance to Tamoxifen treatment, the levels of phosphorylation will decrease. Such a decrease would suggest that Tamoxifen treatment should be halted. A method of the invention can be used to monitor such a decrease in phosphorylation.

Another aspect of the invention is a kit useful for any of the methods disclosed herein. For example, the kit can be useful for predicting the response of a subject suffering from breast cancer to Tamoxifen therapy, comprising reagents for measuring the amount of phosphorylation at Bcl-2 S70, EGFR Y992, and/or Src Y527. The reagents can comprise, e.g., antibodies that are specific for particular unphosphorylated and/or phosphorylated isoforms of each of the three biomarkers that are identified herein. Furthermore, the kit may comprise reagents or devices for preparing a sample (e.g., for collecting a tissue and/or excising a sample from the tissue); for spotting test samples on a suitable surface, such as nitrocellulose strips; for performing immuno-quantitation (e.g., labeled antibodies, or reagents for labeling antibodies); instructions for performing a method of the invention; etc. The components of the kit may, optionally, be packaged in one or more containers.

Optionally, a kit of the invention comprises suitable buffers; one or more containers or packaging material; and/or a label indicating a use for the kit. The reagents of the kit may be in containers in which the reagents are stable, e.g., in lyophilized form or stabilized liquids. The reagents may also be in single use form, e.g., in a form for performing a single assay for one or more of the phosphorylated isoforms of the invention.

Suitable controls for assays of the invention will be evident to the skilled worker. For example, to provide for quality control, each set of proteins tested (e.g. in the form of a protein micro-array) may contain antigen controls, cell lysate controls, and/or a reference lysate. Each patient analyte sample can be normalized to total protein and quantitated in units relative to the reference “printed” on the same array. Each reference and control lysate can be printed in the same dilution series as patient samples and be immunostained at the same time, with identical reagents as the patient samples. All samples can be printed in duplicate or other multiples in, e.g., 4-point dilution curves. Alternatively, a “calibration curve” comprising defined calibrants as discussed elsewhere herein, can be printed with each array.

To provide for quality assurance, samples can be processed and analyzed in real time, e.g. as they are received at a suitable processing facility that meets applicable regulatory standards. Samples may consist of Cytolyte preserved samples. A test set with matched frozen samples can verify the adequacy of specimen preservation. Techniques can be carried out at room temperature. Samples may be obtained by core needle biopsy.

Following the determination of the level of phosphorylation of a marker protein by a method as discussed herein, the values can be reported, e.g. in the form of a panel or suite of values, optionally including data providing a comparison to a baseline value (e.g., positive and/or negative reference standards) to physicians to improve therapy decisions for their patients. With such a report, breast cancers may be stratified at a molecular level, according to whether Tamoxifen therapy is likely to be effective. Some suitable systems for reporting the data are described in co-pending U.S. application Ser. No. 12/057,163, filed Mar. 27, 2008.

In the foregoing and in the following examples, all temperatures are set forth in uncorrected degrees Celsius; and, unless otherwise indicated, all parts and percentages are by weight.

### EXAMPLES

### Example I

### Materials and Methods

Laser Capture Microdissection: 8 um frozen sections were prepared on either glass or membrane slides. Frozen sections were fixed in 70% ethanol, stained with Mayer's Hematoxylin and Scott's Tap Water Substitute, and dehydrated in gradient ethanol, with a final clearing in xylene. The slides were rapidly air dried and tumor cells were isolated by laser capture microdissection (Pixcell™ and Veritas™, Arcturus Molecular Devices; CA, USA).

Reverse Phase Protein Microarrays. The microdissected cells were subjected to lysis in 2.5% beta-mercaptoethanol in T-PER (Pierce, Rockford, Ill.) and 2×SDS Tris-glycine buffer (Invitrogen, Carlsbad, Calif.). The protein lysates were loaded into 384-well plates and each serially diluted in Lysis Buffer to a five point dilution curve (neat, ½, ¼, ⅛ and 1/16) as described by Sheehan et al. (2005) supra. Each dilution series was printed in duplicate onto nitrocellulose-coated glass slides (Whatman, Inc., Sanford, Me.) with a 2470 Arrayer (Aushon BioSystems, Burlington, Mass.), outfitted with 350 μm pins, for a final deposited volume of approximately 33 nl per spot. Total protein in each spot ranged from 4 μg to 250 ng. Slides were stored dessicated at −20° C. For estimation of total protein amounts, selected arrays were stained with Sypro Ruby Protein Blot Stain (Molecular Probes, Eugene, Oreg.) according to the manufacturer's instructions and visualized on an Affymetrix 428 Array Scanner (Santa Clara, Calif.). Prior to antibody staining, the lysate arrays were treated with mild Reblot antibody stripping solution (Chemicon, Temecula, Calif.) for 15 min at room temperature, washed two x five min in PBS, and then incubated for at least 5 hours in blocking solution (1 g I-block (Tropix, Bedford, Mass.), 0.1% Tween-20 in 500 mL PBS) at room temperature with constant rocking. The slides were stored with desiccant (Drierite, W.A. Hammond, Xenia, Ohio) at −20° C. prior to immunostaining.

Protein Microarray Immunostaining: Immunostaining was performed on an automated slide stainer per manufacturer's instructions (Autostainer CSA kit, Dako, Carpinteria, Calif.).

### Protein Microarray Staining:

Blocked arrays were stained with antibodies on an automated slide stainer (Dako Cytomation, Carpinteria, Calif.) using the Catalyzed Signal Amplification System kit according to the manufacturer's recommendation (CSA; Dako Cytomation). Briefly, endogenous biotin was blocked for 10 min using the biotin blocking kit (Dako Cytomation), followed by application of protein block for 5 min; primary antibodies were diluted in antibody diluent and incubated at room temperature for 30 minutes. The primary antibodies used included: EGFR Y992, Bcl-2 S70 and Src Y527 (Cell Signaling Technology, Danvers, Mass.). Each slide was then incubated with a goat anti-rabbit IgG H+L (1:5000) (Vector Labs, Burlingame, Calif.) secondary antibody for 15 minutes. The negative control slide was incubated with antibody diluent. Signal amplification involved incubation with a streptavidin-biotin-peroxidase complex provided in the CSA kit for 15 min, and amplification reagent, (biotinyl-tyramide/hydrogen peroxide, streptavidin-peroxidase) for 15 min each. Development was completed using diaminobenzadine/hydrogen peroxide as the chromogen/substrate. Secondary antibodies and dilutions used in this study were: biotinlyated goat anti-rabbit IgG (H+L) 1:5000 (Vector Laboratories, Burlingame, Calif., USA); and biotinylated rabbit anti-mouse IaG 1:10 (Dako Cytomation).

### Image Analysis:

Stained slides were scanned individually on a UMAX PowerLook III scanner (UMAX, Dallas, Tex., USA) at 600 dpi and saved as TIF files in Photoshop 6.0 (Adobe, San Jose, Calif., USA). The TIF images for antibody-stained slides and Sypro-stained slide images were analyzed with MicroVigene image analysis software, version 2.200 (Vigenetech, North Billerica, Mass.) and Microsoft Excel 2000 software. Images were imported into Microvigene which performed spot finding, local background subtraction, replicate averaging and total protein normalization, producing a single value for each sample at each endpoint.

Bioinformatics method for microarray analysis. The Ward method for two-way hierarchical clustering was performed using JMP v5.0 (SAS Institute, Cary N.C.). Wilcoxon two-sample rank sum test was used to compare values between two groups. P values less than 0.05 were considered significant. When we couldn't assume a normal distribution of the variables we used non-parametric methods.

### Example II

### Progression Free-Survival (PFS) and Post-Relapse-Survival (PRS) in Patients with Recurrent Breast Disease Who are Treated with Tamoxifen, as a Function of the Degree of Phosphorylation of BcI-2 S70, Src Y527 and EGFR Y992

Because tissue specimens of recurrent disease are scarce, we conducted proteomic signal pathway profiling of pre-therapy primary tumor tissues from patients treated with adjuvant Tamoxifen upon disease recurrence to identify potential markers that correlate with therapeutic response or survival following recurrence. Approximately 10-15,000 tumor epithelial cells were microdissected from 38 primary breast tumors representing patients with recurrent disease that responded (n=18) or progressed (n=20) during Tamoxifen therapy. Total protein lysates from these cells were subjected to reverse-phase protein microarray analysis to assess the phosphorylation levels of specific residues in about 22 key proteins regulating apoptosis, proliferation, and survival signaling. The 22 endpoints tested were: Akt S473; Bcl2 S70; cAbl T735; cl Casp3 D175; EGFR Y992; EGFR Y1173; eIF4G S1108; ErbB2 &221, 1222; ERK T202, Y204; FAK Y397; GSK 3ab S21, 9; p70S6K T289; p70S6 S371; total ErbB2 b; MEK1, 2 S217, 221, 062306; cKit Y719; STAT3 Y705; EBP1 T70; mTOR S2448; PAK1 (199/204)/PAK2 (S192/197); PKCα S657; and Src Y527.

Mean comparison revealed no significant differences in phosphorylation levels for most of these endpoints between responder and non-responder categories. However, statistically significant correlations between progression-free survival (PFS), post-relapse survival (PRS) and the phosphorylation levels were observed for three of the endpoints. Increased phosphorylation of Bcl-2 S70, EGFR Y992, and Src Y527 were associated with longer PFS and PRS times.

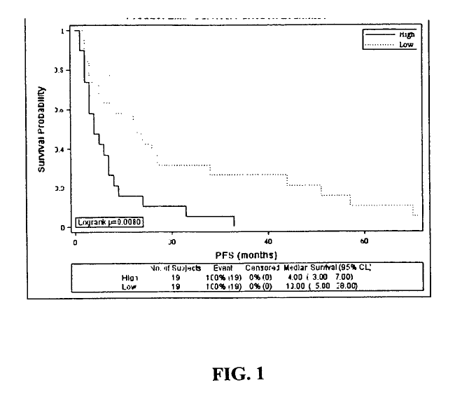

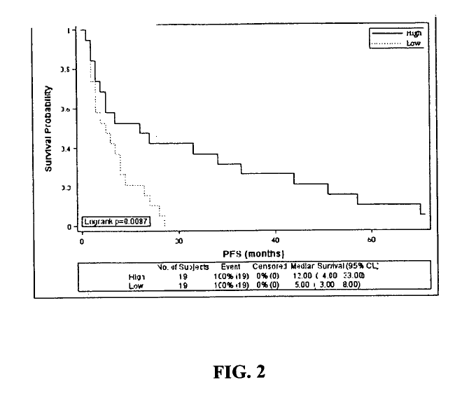

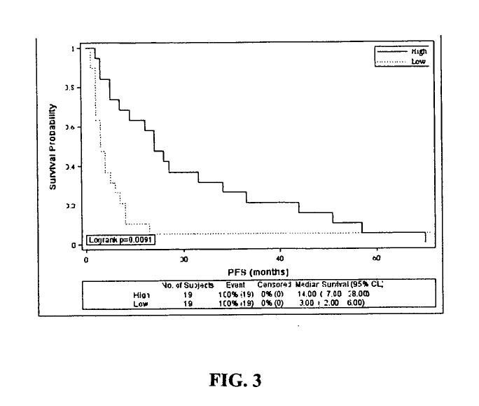

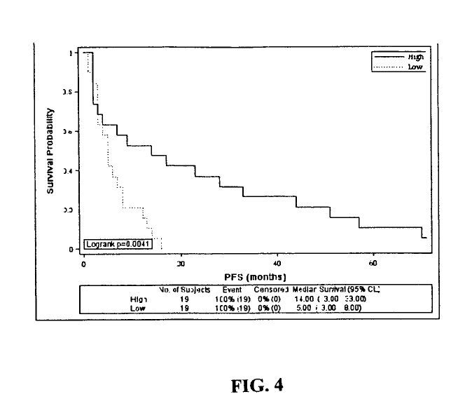

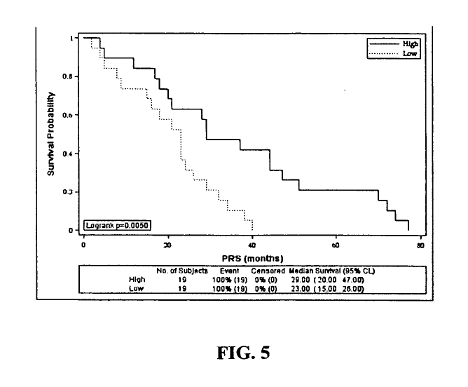

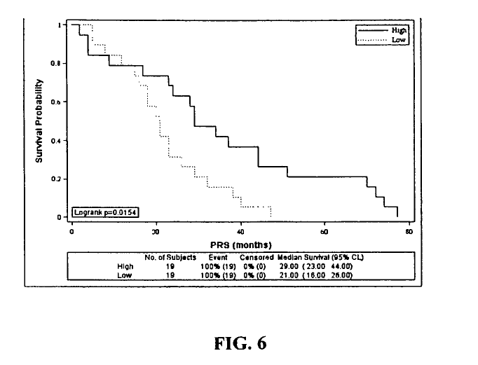

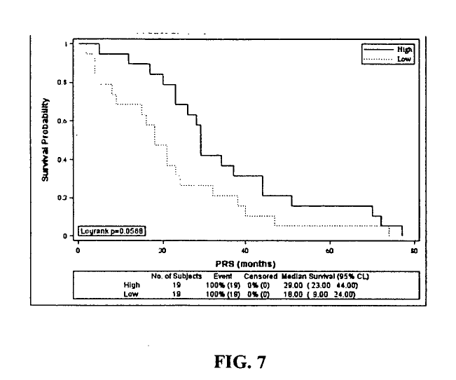

Kaplan-Meier curves for PFS are shown for EGFR Y992, Bcl-2 S70, and Src Y527 in FIGS. 2, 3 and 4, respectively. Kaplan-Meier curves for PRS are shown for EGFR Y992, Bcl-2 S70, and Src Y527 in FIGS. 5, 6 and 7, respectively. FIG. 1 shows a typical example of a Kaplan-Meier curve (PFS) for an endpoint that is not correlated with responsiveness to Tamoxifen: PKCα. The three phosphoprotein biomarkers identified herein provide useful information for rational therapeutic selection of Tamoxifen and improved response rates, e.g. in a recurrent setting.

From the foregoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding preferred specific embodiments are to be construed as merely illustrative, and not limiting of the scope of the invention in any way whatsoever. The entire disclosure of all applications, patents, and publications cited above, including U.S. Provisional application 60/935,106, filed Jul. 26, 2007, and in the figures are hereby incorporated in their entirety by reference.

1.-19. (canceled)

20. A method for predicting the response to Tamoxifen treatment of a human having recurrent, non-metastasizing, estrogen receptor positive (ER⁺) breast cancer, wherein the response is measured by progression-free survival (PFS) and/or post-relapse survival (PRS), comprising:
measuring the amount of phosphorylation at residue S70 of Bcl-2, residue Y992 of EGFR, and/or residue Y527 of Src in a suitable sample from the human, compared to a negative and a positive reference standard,
wherein a statistically significantly elevated level of phosphorylation of one or more of Bcl-2(S70), EGFR(Y992) or Src(Y527) compared to the negative reference standard, or a level that is statistically the same as the positive reference standard, indicates that the human is likely to be responsive to Tamoxifen therapy, and
a statistically significantly reduced level of phosphorylation of one or more of Bcl-2(S70), EGFR(Y992) or Src(Y527) compared to the positive reference standard, or a level that is statistically the same as the negative reference standard, indicates that the human is likely to be non-responsive to Tamoxifen therapy.

21. A method for predicting the prognosis of a human having recurrent, non-metastasizing, ER⁺ breast cancer who is receiving Tamoxifen treatment, comprising measuring the amount of phosphorylation at residue S70 of Bcl-2, residue Y992 of EGFR, and/or residue Y527 of Src in a suitable sample from the human, compared to a negative and a positive reference standard,
wherein a statistically significantly elevated level of phosphorylation of one or more of Bcl-2(S70), EGFR(Y992) or Src(Y527) compared to the negative reference standard, or a level that is statistically the same as the positive reference standard, indicates that the human is likely to have a good prognosis, and
a statistically significantly reduced level of phosphorylation of one or more of Bcl-2(S70), EGFR(Y992) or Src(Y527) compared to the positive reference standard, or a level that is statistically the same as the negative reference standard, indicates that the human is likely to have a poor prognosis.

22. A method for determining a therapeutic treatment regimen for a human having recurrent, non-metastasizing, ER⁺ breast cancer, based upon the human's expected response or lack of response to treatment with Tamoxifen, as measured by PFS or PRS, comprising determining an expected response or non-response to Tamoxifen by the method of claim 20, and selecting an appropriate treatment for a human with the determined expected response.

23. The method of claim 22, wherein the treatment comprises administering an effective amount of Tamoxifen to a human who has been determined to be likely to be responsive to Tamoxifen.

24. The method of claim 22, wherein the treatment comprises administering a treatment other than Tamoxifen treatment to a human who has been determined not to be likely to be responsive to Tamoxifen.

25. The method of claim 24, wherein the treatment other than Tamoxifen treatment comprises treatment with an aromatase inhibitor (AI), radiation, and/or one of the chemotherapeutic agents listed in Table 1.

26. A method for treating a human having recurrent, non-metastasizing, ER⁺ breast cancer, comprising administering to the human an effective amount of Tamoxifen, if a suitable sample from the human is shown to harbor a statistically significantly elevated level of phosphorylation at residues S70 of Bcl-2, Y992 of EGFR, and/or Y527 of Src, compared to a baseline value.

27. A method for treating a human having recurrent, non-metastasizing, ER⁺ breast cancer, comprising
measuring the amount of phosphorylation at Bcl-2 residue S-70, EGFR residue Y992, and/or Src residue Y527 in a suitable sample from the human, compared to a negative and a positive reference standard,
wherein a statistically significantly elevated level of phosphorylation of one or more of Bcl-2(S70), EGFR(Y992) or Src(Y527) compared to the negative reference standard, or a level that is statistically the same as the positive reference standard, indicates that the human is likely to be responsive to Tamoxifen therapy, as measured by PFS or PRS, and
a statistically significantly reduced level of phosphorylation of one or more of Bcl-2(S70), EGFR(Y992) or Src(Y527) compared to the positive reference standard, or a level that is statistically the same as the negative reference standard, indicates that the human is likely to be non-responsive to Tamoxifen therapy, as measured by PFS or PRS; and
if the levels of phosphorylation compared to a baseline value suggest that the human is likely to be responsive to Tamoxifen therapy, administering an effective amount of Tamoxifen to the human, or
if the levels of phosphorylation compared to a baseline value suggest that the human is likely not to be responsive to Tamoxifen therapy, administering a treatment other than Tamoxifen treatment.

28. The method of claim 27, wherein the treatment other than Tamoxifen treatment comprises treatment with an aromatase inhibitor (AI), radiation, and/or one of the chemotherapeutic agents listed in Table 1.

29. A method for monitoring the effectiveness of treating with Tamoxifen a human having recurrent, non-metastasizing, ER⁺ breast cancer, comprising
measuring the amount of phosphorylation at residue S70 of Bcl-2, residue Y992 of EGFR, and/or residue Y527 of Src in a suitable sample from the human, compared to the amount of phosphorylation of the human before Tamoxifen treatment was initiated,
wherein a statistically significantly reduced level of phosphorylation of one or more of Bcl-2(S70), EGFR(Y992) or Src(Y527) compared to the level of phosphorylation before the treatment began indicates that the human is becoming non-responsive to the Tamoxifen therapy.

30. The method of claim 20, wherein the amount of phosphorylation at one or more of the residues is detected by measuring the amount of reactivity of an antibody specific for the phosphorylated isoform of residue S70 of Bcl-2, residue Y992 of EGFR, and/or residue Y527 of Src in the sample.

31. The method of claim 29, wherein the amount of phosphorylation at one or more of the residues is detected by measuring the amount of reactivity of an antibody specific for the phosphorylated isoform of residue S70 of Bcl-2, residue Y992 of EGFR, and/or residue Y527 of Src in the sample.

32. The method of claim 20, which comprises measuring the amount of phosphorylation at two or more of the residues S70 of Bcl-2, Y992 of EGFR, and/or Y527 of Src.

33. The method of claim 29, which comprises measuring the amount of phosphorylation at two or more of the residues S70 of Bcl-2, Y992 of EGFR, and/or Y527 of Src.

34. The method of claim 30, which comprises measuring the amount of phosphorylation at two or more of the residues S70 of Bcl-2, Y992 of EGFR, and/or Y527 of Src.

35. The method of claim 31, which comprises measuring the amount of phosphorylation at two or more of the residues S70 of Bcl-2, Y992 of EGFR, and/or Y527 of Src.

36. The method of claim 20, which comprises measuring the amount of phosphorylation at all three of the residues S70 of Bcl-2, Y992 of EGFR, and Y527 of Src.

37. The method of claim 29, which comprises measuring the amount of phosphorylation at all three of the residues S70 of Bcl-2, Y992 of EGFR, and Y527 of Src.

38. The method of claim 30, which comprises measuring the amount of phosphorylation at all three of the residues S70 of Bcl-2, Y992 of EGFR, and Y527 of Src.

39. The method of claim 31, which comprises measuring the amount of phosphorylation at all three of the residues S70 of Bcl-2, Y992 of EGFR, and Y527 of Src.

40. A kit for predicting the response of a human having recurrent, non-metastasizing, ER⁺ breast cancer to Tamoxifen, comprising reagents for measuring the amount of phosphorylation at one or more of residues S70 of Bcl-2, Y992 of EGFR, and/or Y527 of Src Y1068, optionally in one or more containers.

41. The kit of claim 40, which comprises reagents for measuring the amount of phosphorylation at two or more of residues S70 of Bcl-2, Y992 of EGFR, and/or Y527 of Src Y1068.

42. The kit of claim 40, which comprises reagents for measuring the amount of phosphorylation at all three of residues S70 of Bcl-2, Y992 of EGFR, and Y527 of Src Y1068.

43. A method comprising
obtaining a tissue sample;
obtaining data regarding the levels of phosphorylation of one or more of Bcl-2 S70, EGFR Y992, and/or Src Y527 in the sample; and
providing a report of those phosphorylation levels in which the levels are compared to the levels in a control population.
